(19) United States
(12) Patent Application Publication
(10) Pub. No.: US 2011/0184291 A1
(43) Pub. Date: Jul. 28, 2011

(54) ULTRASONIC DIAGNOSTIC APPARATUS, MEDICAL IMAGE DIAGNOSTIC APPARATUS, ULTRASONIC IMAGE PROCESSING APPARATUS, MEDICAL IMAGE PROCESSING APPARATUS, ULTRASONIC DIAGNOSTIC SYSTEM, AND MEDICAL IMAGE DIAGNOSTIC SYSTEM

(76) Inventors: Yoko OKAMURA, Nasushiobara-shi (JP); Naohisa KAMIYAMA, Otawara-shi (JP)

(21) Appl. No.: 13/014,219

(22) Filed: Jan. 26, 2011

(30) Foreign Application Priority Data
Jan. 27, 2010 (JP) 2010-015891
Jan. 24, 2011 (JP) 2011-011730

(51) Int. Cl. A61B 8/14

(52) U.S. Cl. 600/443

(57) ABSTRACT

According to one embodiment, an ultrasonic diagnostic apparatus comprises a data acquisition unit configured to ultrasonically scan a three-dimensional area including a predetermined region of an object and acquire volume data associated with the three-dimensional area, a calculation unit configured to cut the volume data at at least one plane and calculate a full-scale contour of a slice of the predetermined region and a full-scale planned surgical line used for surgical operation of the predetermined region, and an output unit configured to output at least one of the full-scale contour of the slice of the predetermined region and the full-scale planned surgical line.

### CROSS-REFERENCE TO RELATED APPLICATIONS

This application is based upon and claims the benefit of priority from Japanese Patent Applications No. 2010-015891, filed Jan. 27, 2010; and No. 2011-011730, filed Jan. 24, 2011; the entire contents of both of which are incorporated herein by reference.

### FIELD

Embodiments described herein relate generally to an ultrasonic diagnostic apparatus, medical image diagnostic apparatus, ultrasonic image processing apparatus, medical image processing apparatus, ultrasonic diagnostic system, and medical image diagnostic system which are used when an operation target region or a treatment target region is to be marked before surgical operation or treatment.

### BACKGROUND

An ultrasonic diagnostic apparatus can display in real time how the heart beats or the fetus moves, by simply bringing an ultrasonic probe into contact with the body surface. This apparatus is highly safe, and hence allows repetitive examination. Furthermore, this system is smaller in size than other diagnostic apparatuses such as X-ray, CT, and MRI apparatuses and can be moved to the bedside to be easily and conveniently used for examination. In addition, the ultrasonic diagnostic apparatus is free from the influences of exposure using X-rays and the like, and hence can be used in obstetric treatment, treatment at home, and the like.

In addition, such an ultrasonic diagnostic apparatus, owing to its high real-time performance, is used not only for image diagnosis but also for support before or during surgical operation. For example, it is possible to make a surgical plan including an incision method by re-checking a lesion to be excised before surgical operation and checking the positions of surrounding blood vessels and the like using ultrasonic images. This apparatus is often used for marking of a planned surgical line especially in breast cancer operation or the like.

In this case, an operator executes marking to determine a place to be incised immediately before surgical operation, by drawing the position and size of a tumor (lesion or the like) or a planned surgical line on the body surface (breast surface) with an inkpen (note that the operator cannot acquire precise depth information). In addition, the operator marks an incision region, an approach method, and the like on the body surface. Under present circumstances, an operator marks a tumor shape while acquiring and checking an ultrasonic image of the periphery of a lesion several ten times.

Conventionally, however, when marking a lesion and a planned surgical line before surgical operation, the operator needs to acquire an ultrasonic image of the periphery of a lesion several ten times and accurately check the periphery of the lesion with caution. For this reason, marking takes much time and labor, and hence leads to a deterioration in operation efficiency at the time of surgical operation.

### BRIEF DESCRIPTION OF THE DRAWINGS

FIG. 1 is a block diagram showing the arrangement of an ultrasonic diagnostic apparatus according to the first embodiment;

FIG. 2 is a flowchart showing a procedure for processing (planned surgical line marking support processing) based on a planned surgical line marking support function according to this embodiment;

FIG. 3 is a view showing an example of a VR image with a scanning slice position including information (position marker) indicating a position on a volume rendering image;

FIG. 4 is a view showing an example of how a sheet on which a full-scale planned surgical line is printed is pasted on the body surface;

FIG. 5 is a view for explaining an output form according to the first modification;

FIG. 6 is a view for explaining an output form according to the second modification;

FIG. 7 is a flowchart showing a procedure for planned surgical line marking support processing according to the third embodiment; and

FIG. 8 is a block diagram for explaining an ultrasonic diagnostic system S according to the fourth embodiment.

### DETAILED DESCRIPTION

In general, according to one embodiment, there is provided an ultrasonic diagnostic apparatus comprising a data acquisition unit configured to ultrasonically scan a three-dimensional area including a predetermined region of an object and acquire volume data associated with the three-dimensional area, a calculation unit configured to cut the volume data at at least one plane and calculate a full-scale contour of a slice of the predetermined region and a full-scale planned surgical line used for surgical operation of the predetermined region, and an output unit configured to output at least one of the full-scale contour of the slice of the predetermined region and the full-scale planned surgical line.

An embodiment will be described below with reference to the views of the accompanying drawing. Note that the same reference numerals in the following description denote constituent elements having almost the same functions and arrangements, and a repetitive description will be made only when required. For the sake of a concrete description, assume that a diagnostic target is a breast in each embodiment. However, the embodiments are not limited to this, and each technical idea of the present embodiments is effective for predetermined organs other than the breasts, e.g., the liver and the pancreas.

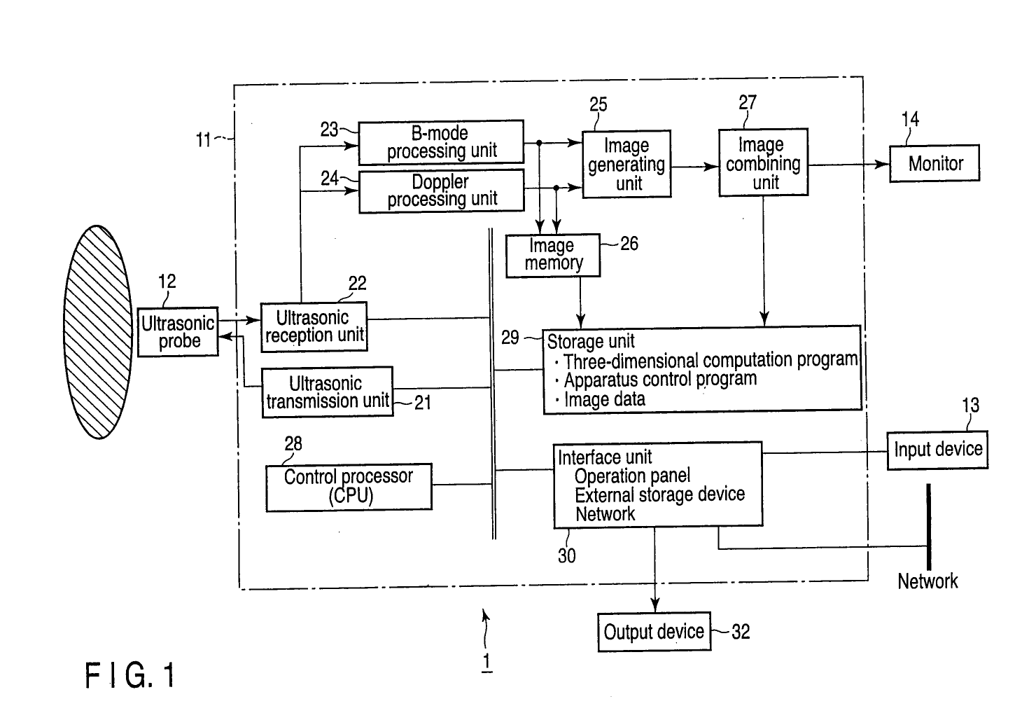

FIG. 1 is a block diagram showing the arrangement of an ultrasonic diagnostic apparatus 1 according to this embodiment. As shown in FIG. 1, the ultrasonic diagnostic apparatus 1 includes an apparatus body 11, an ultrasonic probe 12, an input device 13, a monitor 14, and an output device 32 connected to the apparatus body 11 as needed.

The ultrasonic probe 12 includes a plurality of piezoelectric transducers which generate ultrasonic waves based on driving signals from the apparatus body 11 and convert reflected waves from an object into electrical signals, a matching layer provided for the piezoelectric transducers, and a backing member which prevents ultrasonic waves from propagating backward from the piezoelectric transducers. When the ultrasonic probe 12 transmits an ultrasonic wave to an object, the transmitted ultrasonic wave is sequentially reflected by a discontinuity surface of acoustic impedance of internal body tissue, and is received as an echo signal by the ultrasonic probe 12. The amplitude of this echo signal depends on an acoustic impedance difference on the discontinuity surface by which the echo signal is reflected. The echo produced when a transmitted ultrasonic pulse is reflected by the surface of a moving blood flow, cardiac wall, or the like is subjected to a frequency shift depending on the velocity component of the moving body in the ultrasonic transmission direction due to the Doppler effect.

Assume that the ultrasonic probe 12 is a swinging probe or two-dimensional array probe which can ultrasonically scan a three-dimensional area. A swinging probe can perform ultrasonic scanning while mechanically swinging a plurality of ultrasonic transducers arrayed in a predetermined direction along a direction perpendicular to the array direction. A two-dimensional array probe includes a plurality of ultrasonic transducers arrayed in a two-dimensional matrix, and can three-dimensionally control the transmitting and receiving directions of ultrasonic beams.

The input device 13 is connected to an apparatus body 11 and includes various types of switches, buttons, a trackball, a mouse, and a keyboard which are used to input, to the apparatus body 11, various types of instructions, conditions, an instruction to set a region of interest (ROI), various types of image quality condition setting instructions, and the like from an operator. When, for example, the operator operates the end button or FREEZE button of the input device 13, the transmission/reception of ultrasonic waves is terminated, and the ultrasonic diagnostic apparatus is set in a pause state.

The monitor 14 displays morphological information and blood flow information in the living body as images based on video signals from an image generating unit 25.

The output device 32 includes a printer, projector, and laser output device which output, in predetermined forms, the actual size of a lesion, a planned surgical line, and the like acquired in processing based on a planned surgical line marking support function (to be described later).

The apparatus body 11 includes an ultrasonic transmission unit 21, an ultrasonic reception unit 22, a B-mode processing unit 23, a Doppler processing unit 24, an image generating unit 25, an image memory 26, an image combining unit 27, a control processor (CPU) 28, a storage unit 29, and an interface unit 30.

The ultrasonic transmission unit 21 includes a trigger generating circuit, delay circuit, and pulser circuit (none of which are shown). The pulser circuit repetitively generates rate pulses for the formation of transmission ultrasonic waves at a predetermined rate frequency fr Hz (period: 1/fr sec). The delay circuit gives each rate pulse a delay time necessary to focus an ultrasonic wave into a beam and determine transmission directivity for each channel. The trigger generating circuit applies a driving pulse to the probe 12 at the timing based on this rate pulse.

The ultrasonic transmission unit 21 has a function of instantly changing a transmission frequency, transmission driving voltage, or the like to execute a predetermined scan sequence in accordance with an instruction from the control processor 28. In particular, the function of changing a transmission driving voltage is implemented by linear amplifier type transmission circuit capable of instantly switching its value or a mechanism of electrically switching a plurality of power supply units.

The ultrasonic reception unit 22 includes an amplifier circuit, A/D converter, and adder (none of which are shown). The amplifier circuit amplifies an echo signal received via the probe 12 for each channel. The A/D converter gives the amplified echo signals delay times necessary to determine reception directivities. The adder then performs addition processing for the signals. With this addition, a reflection component from a direction corresponding to the reception directivity of the echo signal is enhanced to form a composite beam for ultrasonic transmission/reception in accordance with reception directivity and transmission directivity.

The B-mode processing unit 23 receives an echo signal from the ultrasonic reception unit 22, and performs logarithmic amplification, envelope detection processing, and the like for the signal to generate data whose signal intensity is expressed by a luminance level. The image generating unit 25 causes the monitor 14 to display, as a B-mode image, a signal from the B-mode processing unit 23 whose reflected wave intensity is expressed by a luminance. At this time, this apparatus can provide image quality suiting user's taste by applying various image filters for edge enhancement, temporal smoothing, spatial smoothing, and the like to the signal.

The Doppler processing unit 24 frequency-analyzes velocity information from the echo signal received from the ultrasonic reception unit 22 to extract a blood flow, tissue, and contrast medium echo component by the Doppler effect, and obtains blood flow information such as an average velocity, variance, and power at multiple points. The obtained blood flow information is sent to the image generating circuit 25, and is displayed in color as an average velocity image, a variance image, a power image, and a combined image of them on the monitor 14.

The image generating unit 25 generates an ultrasonic diagnostic image as a display image by converting the scanning line signal string for ultrasonic scanning into a scanning line signal string in a general video format typified by a TV format. The image generating unit 25 generates a scanning slice image, MPR image, volume rendering image, and the like in accordance with instructions from the input device 13. Furthermore, in processing (planned surgical line marking support processing) based on the planned surgical line marking support function (to be described later), the image generating unit 25 cuts an operation target region (corresponding data) at a plurality of parallel C planes in acquired volume data, and generates a plurality of images corresponding to the respective C slices. Note that data before it is input to the image generating unit 25 is sometimes called “raw data”.

The image memory 26 is a memory to store, for example, ultrasonic images corresponding to a plurality of frames immediately before a freeze. Continuously displaying (cine-displaying) images stored in the image memory 26 can display an ultrasonic moving image.

The image combining unit 27 combines the image received from the image generating unit 25 with character information of various types of parameters, scale marks, and the like, and outputs the resultant signal as a video signal to the monitor 14.

The control processor 28 has the function of an information processing apparatus (computer) and controls the operation of the main body of this ultrasonic diagnostic apparatus. The control processor 28 reads out a control program for executing image generation/display, a dedicated program for implementing a planned surgical line marking support function (to be described later), and the like from a storage unit 29, expands the program in its own memory, and executes computation, control, and the like associated with each type of processing.

The storage unit 29 stores transmission/reception conditions, control programs for executing image generation and display processing, diagnostic information (patient ID, findings by doctors, and the like), a diagnostic protocol, a body mark generation program, a dedicated program for implementing the planned surgical line marking support function (to be described later), and other data. The storage unit 29 is also used to store images in the image memory 26, as needed. It is possible to transfer data in the storage unit 29 to an external peripheral device via the interface unit 30.

The interface unit 30 is an interface associated with the input device 13, a network, and an external storage device. The interface unit 30 can transfer via a network data such as ultrasonic images, analysis results, and the like obtained by this apparatus to another apparatus.

### (Planned Surgical Line Marking Support Function)

The planned surgical line marking support function of the ultrasonic diagnostic apparatus 1 will be described next. This function supports marking of an operation target region at the time of surgical operation by calculating the full-scale contour of a slice of an operation target region (a lesion, focus, or the like) of an object or a planned surgical line with a predetermined margin being added to the full-scale contour, and outputting at least one of them in actual size.

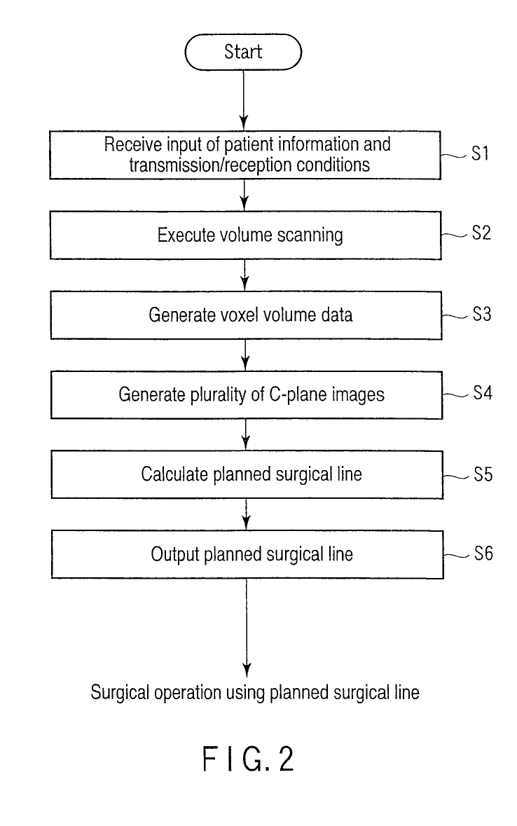

FIG. 2 is a flowchart showing a procedure for processing (planned surgical line marking support processing) based on the planned surgical line marking support function according to this embodiment. Planned surgical line marking support processing will be described with reference to FIG. 2.

### [Input of Patient Information and the Like: Step S1]

First of all, the operator inputs patient information, transmission/reception conditions (a focal depth, transmission voltage, field angle, swinging range, and the like), and the like via the input device 13. The field angle, swinging range, and the like are set to include an operation target region. The control processor 28 stores various kinds of information and conditions in the storage unit 29 (step S1).

### [Execution of Volume Scanning: Step S2]

If, for example, the ultrasonic probe 12 is a swinging probe, the control processor 28 then executes volume scanning on a three-dimensional area including the operation target region by transmitting ultrasonic waves to the respective slices corresponding to a plurality of swinging angles (swinging positions) and receiving the reflected waves while swinging an ultrasonic transducer array in a direction perpendicular to the array direction (step S2). Alternatively, if the ultrasonic probe 12 is a two-dimensional array probe having ultrasonic transducers arrayed in a two-dimensional matrix, volume scanning on a three-dimensional area including the operation target region is executed by three-dimensionally scanning ultrasonic beams.

The echo signal acquired for each slice in step S2 is sent to the B-mode processing unit 23 via the ultrasonic reception unit 22. The B-mode processing unit 23 performs logarithmic amplification, envelope detection processing, and the like for the signal to generate luminance data whose signal intensity is expressed by a luminance level. The image generating unit 25 generates a two-dimensional image (scanning slice image) corresponding to each scanning slice by using the luminance data received from the B-mode processing unit 23.

### [Image Reconstruction (Generation of Volume Data): Step S3]

The image generating unit 25 reconstructs volume data by executing coordinate conversion of a plurality of generated scanning slice image data from the actual spatial coordinate system (i.e., the coordinate system in which the plurality of scanning slice image data are defined) to a volume data spatial coordinate system and performing interpolation processing (step S3).

[Generation of Plurality of C-plane images: Step S4]

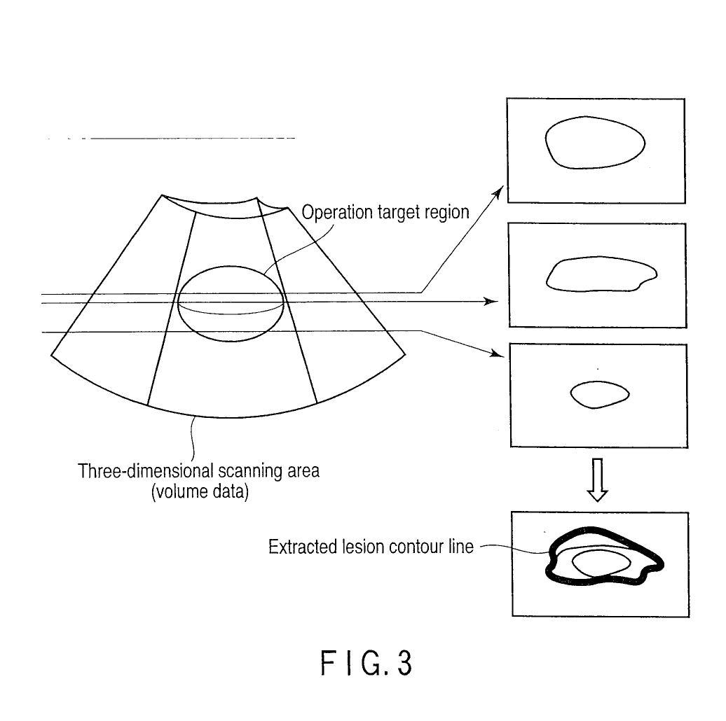

The image generating unit 25 generates a plurality of C-plane images by using the generated volume data (step S4). That is, as shown in FIG. 3, the image generating unit 25 cuts, for example, the operation target region (corresponding data) in the volume data at a plurality of parallel C planes, and generates a plurality of C-plane images corresponding to the respective C slices (step S4).

### [Calculation of Planned Surgical Line: Step S5]

The control processor 28 then calculates a full-scale planned surgical line by using the plurality of generated C-plane images (step S5). For example, as shown on the right side in FIG. 3, the control processor 28 calculates the contour of the operation target region on each generated MPR image, and calculates the full-scale contour of an operation target region slice by using the largest contour line obtained by the AND of the respective calculated contours. In addition, the control processor 28 calculates, as a full-scale planned surgical line, a contour with a margin of a predetermined width being added to the calculated full-scale contour of the operation target region slice.

Note that the method of calculating the full-scale contour of an operation target region slice is not limited to the above example. Another example is to calculate the area of a slice of an operation target region on each generated C-plane image, determine one of the slices of the operation target region which has the largest area, and calculate the full-scale contour of the operation target region slice by using the determined slice. The control processor 28 may also calculate, as a full-scale planned surgical line, a contour with a margin of a predetermined width being added to the calculated full-scale contour of the operation target region slice. Furthermore, the user may determine the width of a margin to be added to the full-scale contour of an operation target region slice for each calculation, or it is possible to use a recommended value stored in the apparatus in advance.

The cutting planes at which the operation target region (corresponding data) in volume data is cut are not limited to C planes. For example, it is possible to set an arbitrary cutting plane (MPR plane) in volume data in response to an input from the operator or automatically. When such a cutting plane is set, the contour of the operation target region on the cutting plane and a planned surgical line are calculated as the actual size of a C-plane image.

### [Output of Planned Surgical Line: Step S6]

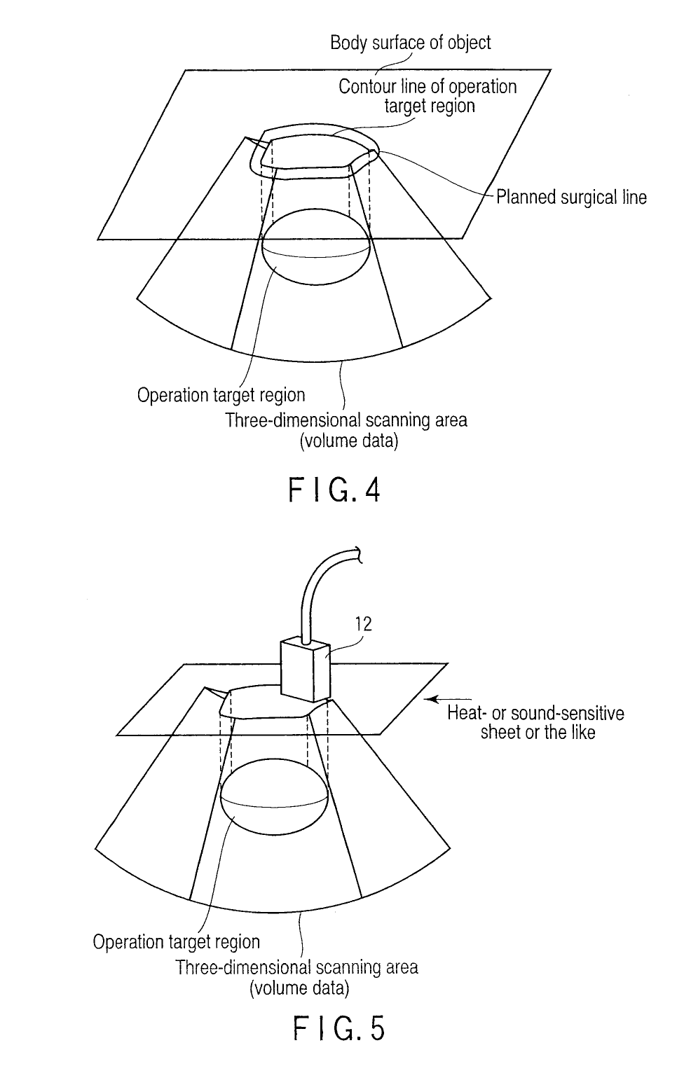

The output device 32 then outputs the calculated full-scale planned surgical line in a predetermined form (step S6). Assume that in this embodiment, the output device 32 prints the planned surgical line on a sheet which can be pasted on the body surface of an object. At the same time, the output device 32 also prints a reference marker as a reference indicating at which position on the body surface the sheet which can be pasted is to be pasted. It is possible to use, as this reference marker, the current position at which the ultrasonic probe 12 is placed on the body surface. The operator pastes the output sheet on the body surface of the object as shown in, for example, FIG. 4 so as to match the current position of the ultrasonic probe 12 with the reference marker, thereby simply and quickly marking a lesion, a planned surgical line, and the like.

Note that the sheet onto which a planned surgical line is to be output is not limited to the one which can be pasted on the body surface of an object. For example, the same effect can be obtained by outputting a full-scale planned surgical line onto trace paper, placing it with reference to a reference marker, and copying the full-scale planned surgical line down on the body surface.

In addition, it is possible to output not only a planned surgical line but also the full-scale contour of an operation target region slice, as needed. Alternatively, it is possible to output only the full-scale operation target region slice. It is possible to arbitrarily select which information is to be output in accordance with, for example, an instruction from the input device 13.

Note that the output form of a planned surgical line is not limited to the above example, and various kinds of output forms are conceivable. Output form variations will be described below with reference to the following embodiments.

### First Modification

An output form according to this modification is that a planned surgical line is output (drawn) onto a heat-sensitive sheet (sound-sensitive sheet) placed between the ultrasonic probe 12 and an object.

FIG. 5 is a view for explaining an output form according to the first modification. As shown in FIG. 5, the operator places a heat-sensitive sheet (sound-sensitive sheet) between the ultrasonic probe 12 and the body surface of the object. The control processor 28 determines transmission conditions such as a beam direction or a sound pressure to draw the contour of a planned surgical line, and controls the ultrasonic transmission unit 21 in accordance with the determined transmission conditions. The ultrasonic beam transmitted under the control of the control processor 28 then draws the planned surgical line on the heat-sensitive sheet (or sound-sensitive sheet).

In order to draw a planned surgical line having a wide range on a heat-sensitive sheet (or sound-sensitive sheet), it is necessary to move the ultrasonic probe 12 along the body surface. In this case, positioning acquired volume data relative to a two-dimensional image acquired at the current position of the ultrasonic probe 12 can determine the direction in which the ultrasonic probe 12 is to be moved. In addition, it is preferable to support the operator in moving the ultrasonic probe 12 by displaying, on the monitor 14 or the like, the determined direction in which the ultrasonic probe 12 is to be moved.

### Second Modification

An output form according to this embodiment is configured to output (project) a planned surgical line on the body surface of an object by using a projector (video projection apparatus).

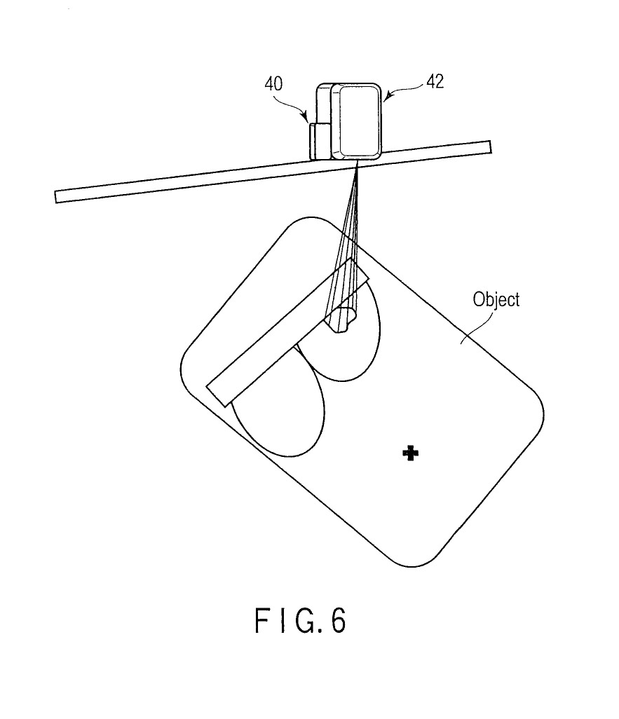

FIG. 6 is a view for explaining the output form according to the second modification. As shown in FIG. 6, for example, a sensor 40 placed immediately above the bed on which an object is placed measures the current position of the ultrasonic probe 12 in real time. The position of the ultrasonic probe 12 measured by the sensor 40 is sequentially transferred to a projector 42. The projector 42 projects the full-scale planned surgical line acquired from the control processor 28 via the interface unit 30 onto the body surface of the object with reference to the transferred position of the ultrasonic probe 12.

### Third Modification

An output form according to this modification is configured to output (project) a planned surgical line onto the body surface of an object by using a laser or the like which does not damage the living body. In the third modification, it is possible to draw a planned surgical line calculated from acquired volume data on the body surface at a position corresponding to the position of the planned surgical line by using an ultrasonic probe including a laser function. In addition, in the third modification as well, the sensor 40 measures the current position of the ultrasonic probe 12 in real time. The position of the ultrasonic probe 12 measured by the sensor 40 is sequentially transferred to a laser output device. The laser output device projects the full-scale planned surgical line acquired from the control processor 28 via the interface unit 30 onto the body surface of the object with reference to the transferred position of the ultrasonic probe 12.

### Effects

According to the above arrangements, the following effects can be obtained.

This ultrasonic diagnostic apparatus performs volume scanning of a three-dimensional area including an operation target region of an object to acquire volume data. This apparatus generates a plurality of C-slice images by using the acquired volume data, and calculates the largest contour or the like of the operation target region. The apparatus calculates the full-scale contour of a slice of the operation target region or a planned surgical line determined upon addition of a predetermined margin to the full-scale contour by using the calculated largest contour or the like, and outputs the resultant information in actual size. The operator can therefore quickly and easily execute marking of an operation target region shape at the time of surgical operation, and can quickly starts surgical operation by using the marked full-scale contour or planned surgical line. This obviates the necessity to perform marking several ten times while repeatedly changing the position of the ultrasonic probe and checking an operation target region. It is therefore possible to reduce the operation load in marking of an operation target region shape at the time of surgical operation.

In addition, this apparatus calculates and outputs the full-scale contour of an operation target region and a planned surgical line by using an ultrasonic image. This can implement marking of an operation target region shape with higher accuracy than that in the prior art, and hence can contribute to an improvement in the quality of medical work.

Furthermore, the apparatus can output the full-scale contour of an operation target region and a planned surgical line in various forms including drawing them on a sheet to be pasted on the body surface of the object, drawing them on a heat-sensitive sheet or sound-sensitive sheet placed between the object and the ultrasonic probe, projecting images of them on the body surface of the object, and projecting them on the body surface of the object using a laser or the like which does not damage the living body. It is therefore possible to select a desired output form in accordance with a surgical operation environment, an object, and the characteristics of an operator and to easily and quickly perform marking of an operation target region shape at the time of surgical operation.

### Second Embodiment

The second embodiment is applied to a medical image diagnostic apparatus (e.g., an X-ray diagnostic apparatus, X-ray computed tomography apparatus, magnetic resonance imaging apparatus, and nuclear medicine diagnostic apparatus) configured to perform imaging upon placing an object on a bed.

These apparatuses also acquire volume data of a three-dimensional area including an operation target region and calculate a planned surgical line or the like by almost the same method as that in the first embodiment. The planned surgical line or the like obtained by calculation is in one of the output forms according to the first embodiment and the respective modifications.

At this time, such an apparatus outputs a sheet to be pasted on the body surface of an object or projects a planned surgical line on the body surface using a projector or a laser with reference to a predetermined position on the bed (e.g., the top). That is, it is possible to easily define a scanning range for the object on the bed (i.e., the acquisition range of volume data) as a three-dimensional coordinate system on the top of the bed. Therefore, the apparatus prints a reference marker together with a planned surgical line as a marker for placing a full-scale planned surgical line at a predetermined position in the three-dimensional coordinate system on the top of the bed in, for example, a form of matching the marker with the predetermined reference position on the top of the bed. It is also possible to project a full-scale planned surgical line on the body surface of an object using a projector or a laser based on a position on volume data or a position on the body surface in the three-dimensional coordinate system on the top of the bed.

The above arrangement can acquire the same effects as those of the first embodiment by using a medical image diagnostic apparatus.

### Third Embodiment

The first and second embodiments are configured to generate voxel volume data and then extract the contour of an operation target region on each of a plurality of C-plane images obtained by cutting the voxel volume data. In contrast to this, the planned surgical line marking support function of the third embodiment extracts the contour of an operation target region or the like on voxel volume data and cuts the voxel volume data at an arbitrary slice, thereby calculating and outputting a full-scale planned surgical line on an MPR image corresponding to the arbitrary slice.

For the sake of a concrete description, consider the planned surgical line marking support function according to this embodiment in the ultrasonic diagnostic apparatus. However, the third embodiment is not limited to this, and can be applied to a medical image diagnostic apparatus which performs imaging after an object is placed on the bed, as in the second embodiment.

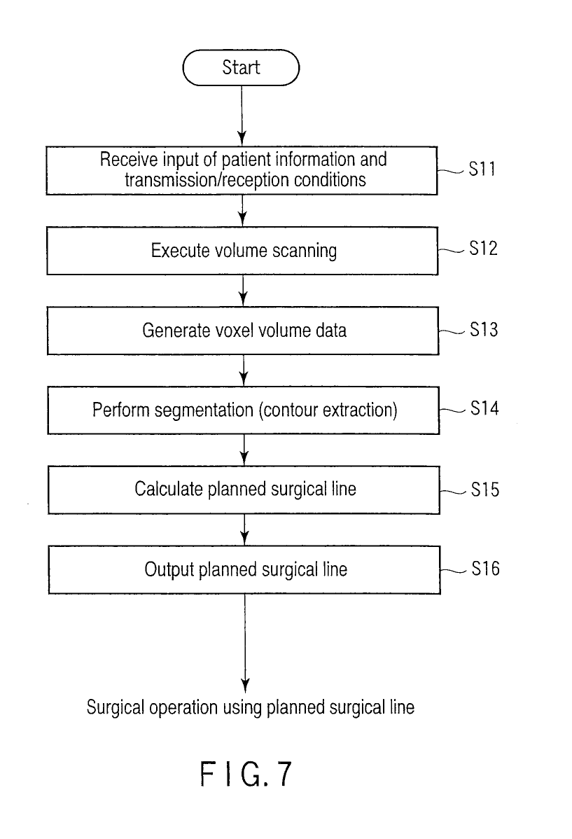

FIG. 7 is a flowchart showing a procedure for planned surgical line marking support processing according to this embodiment. Planned surgical line marking support processing will be described with reference to FIG. 7. Note that steps S11 to S13 are almost the same as steps S1 to S3 in FIG. 2. The contents of processing in each of steps S14 to S17 will therefore be described below.

### [Segmentation (Contour Extraction): Step S14]

An image generating unit 25 executes segmentation processing (area extraction processing) for the generated volume data to extract the contour of the operation target region of the object (step S14). It is possible to implement this segmentation processing by any methods. Typically, it is possible to use, for example, a method of extracting voxels having voxel values larger than a predetermined value by threshold processing.

### [Calculation of Planned Surgical Line: Step S15]

The image generating unit 25 sets an arbitrary cutting plane in the volume data from which the contour of the operation target region has been extracted, and calculates the full-scale contour of the operation target region slice when the cutting plane is projected on a C plane, and a full-scale planned surgical line with a margin of a predetermined width being added to the contour (step S15). Note that a cutting plane is not limited to a plane parallel to a C plane, and is set at a predetermined position in the volume data in response to an input from the operator or automatically. When a cutting plane is automatically set, it is preferable to set the cutting plane so as to cut the operation target region with a maximum area. For example, it is possible to set a cutting plane by a method of calculating the center of gravity of an extracted operation target region, calculating a plane including a circle (or an ellipse) of circles (or ellipses) inscribed in or circumscribed around the operation target region centered on the center of gravity which has the largest diameter (or long axis), and setting the plane as a cutting plane.

### [Output of Planned Surgical Line: Step S16]

The output device 32 outputs the calculated full-scale planned surgical line in a predetermined form (step S16). Output form variations for full-scale planned surgical lines have been described above.

The arrangement described above can also acquire the same effects as those of the first embodiment. In this embodiment, in particular, the apparatus sets an arbitrary cutting plane in volume data, and projects and outputs the contour of an operation target region on the cutting plane and a planned surgical line onto a C plane. Therefore, it is possible to reflect the largest diameter of an operation target region in a contour or planned surgical line to be output. This makes it possible to perform marking with higher accuracy and safety.

### Fourth Embodiment

This embodiment implements the planned surgical line marking support function according to any one of the first to third embodiments by using an ultrasonic diagnostic system including an ultrasonic diagnostic apparatus and an ultrasonic image processing apparatus, and a medical image diagnostic system including a medical image diagnostic apparatus and a medical image processing apparatus. For the sake of a concrete description, consider a case in which the embodiment is implemented by an ultrasonic diagnostic system including an ultrasonic diagnostic apparatus and an ultrasonic image processing apparatus.

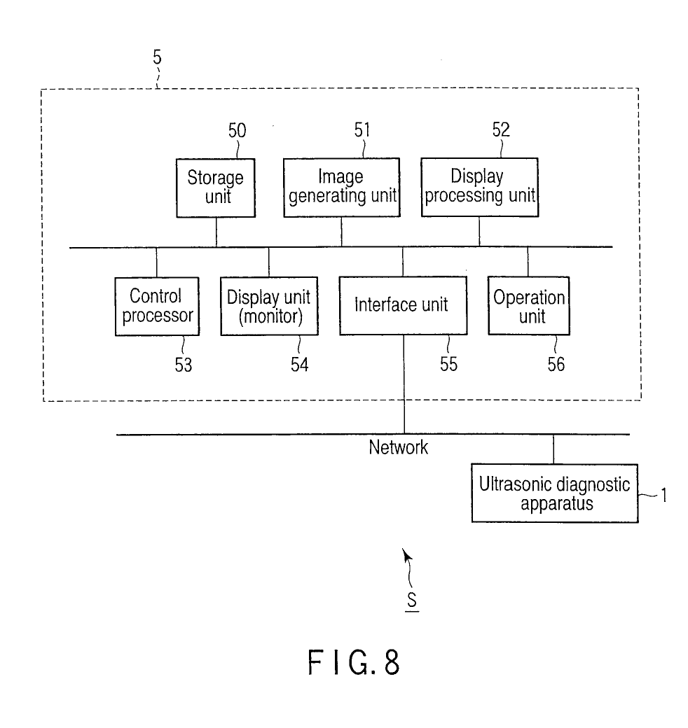

FIG. 8 is a block diagram for explaining an ultrasonic diagnostic system S including an ultrasonic diagnostic apparatus 1 and an ultrasonic image processing apparatus 5. As shown in FIG. 8, the ultrasonic image processing apparatus 5 is implemented by, for example, a medical workstation, and includes a storage unit 50, an image generating unit 51, a display processing unit 52, a control processor 53, a display unit 54, an interface unit 55, and an operation unit 56.

The storage unit 50 stores ultrasonic images acquired in advance and ultrasonic images transmitted from the ultrasonic diagnostic apparatus 1 via a network. The image generating unit 51 executes the planned surgical line marking support processing described above. The display processing unit 52 executes various kinds of processes associated with a dynamic range, luminance (brightness), contrast, γ curve correction, RGB conversion, and the like for various kinds of image data generated/processed by the image processing unit 50. The control processor 53 reads out a dedicated program for implementing the planned surgical line marking support function described above, expands the program in its own memory, and executes computation/control and the like associated with various kinds of processes. The display unit 54 is a monitor to display an ultrasonic image or the like in a predetermined form. The interface unit 55 is an interface for network connection and connection to other external storage devices. The operation unit 56 includes switches, buttons, a trackball, a mouse, and a keyboard which are used to input various types of instructions to the apparatus.

When performing the planned surgical line marking support processing shown in FIG. 2 by using the ultrasonic diagnostic system S, the ultrasonic diagnostic apparatus 1 executes, for example, the processes in steps S1 and S2, and the ultrasonic image processing apparatus 5 executes the processes in steps S3 to S6. Alternatively, the ultrasonic diagnostic apparatus 1 can execute the processes in steps S1 to S3, and the ultrasonic image processing apparatus 5 can execute the processes in steps S4 to S6.

Likewise, when performing the planned surgical line marking support processing shown in FIG. 7 using the ultrasonic diagnostic system S, the ultrasonic diagnostic apparatus 1 executes the processes in steps S11 and S12, and the ultrasonic image processing apparatus 5 executes the processes in steps S13 to S17. Alternatively, the ultrasonic diagnostic apparatus 1 can execute the processes in steps S11 to S13, and the ultrasonic image processing apparatus 5 can execute the processes in steps S14 to S17.

The above arrangement can also acquire the effects described in the first to third embodiments.

Note that the present embodiment is not limited to each embodiment described above, and constituent elements can be modified and embodied in the execution stage within the spirit and scope of the embodiment.

The following are concrete modifications.

(1) Each function (each function in planned surgical line marking support) associated with each embodiment can also be implemented by installing programs for executing the corresponding processing in a computer such as a workstation and expanding them in a memory. In this case, the programs which can cause the computer to execute the corresponding techniques can be distributed by being stored in recording media such as magnetic disks ((floppy®) disks, hard disks, and the like), optical disks (CD-ROMs, DVDs, and the like), and semiconductor memories.

(2) Each embodiment described above has exemplified the case in which planned surgical line marking is supported. However, the technical idea of the present embodiment is not limited to the techniques used for surgical operation, and can be used in a case in which, for example, when a radiation treatment apparatus treats a lesion by irradiating it with radiation, an irradiation range is marked.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
a data acquisition unit configured to ultrasonically scan a three-dimensional area including a predetermined region of an object and acquire volume data associated with the three-dimensional area;
a calculation unit configured to cut the volume data at at least one plane and calculate a full-scale contour of a slice of the predetermined region and a full-scale planned surgical line used for surgical operation of the predetermined region; and
an output unit configured to output at least one of the full-scale contour of the slice of the predetermined region and the full-scale planned surgical line.

2. The apparatus according to claim 1, wherein the calculation unit extracts a contour of the predetermined region included in the at least one plane, and calculates the full-scale contour of the predetermined region slice and the full-scale planned surgical line by using the extracted contour of the predetermined region.

3. The apparatus according to claim 1, wherein the calculation unit sets the plurality of planes, extracts the contour of the predetermined region included in each of the planes, and calculates the full-scale contour of the predetermined region slice by using a largest plane of the contours of the predetermined region extracted on the respective planes.

4. The apparatus according to claim 3, wherein the calculation unit sets the plurality of planes to be substantially parallel to each other.

5. The apparatus according to claim 1, wherein the calculation unit extracts the contour of the predetermined region in the volume data, and calculates the full-scale contour of the predetermined region slice and the full-scale planned surgical line by using the extracted contour at which the at least one plane is cut.

6. The apparatus according to claim 1, wherein the calculation unit calculates the full-scale planned surgical line by using the full-scale contour and a margin amount of a predetermined width.

7. The apparatus according to claim 1, wherein the output unit prints at least one of the full-scale planned surgical line and the full-scale contour onto a sheet configured to be pasted on the body surface of the object.

8. The apparatus according to claim 1, wherein the output unit projects at least one of the full-scale planned surgical line and the full-scale contour onto the body surface of the object.

9. The apparatus according to claim 8, wherein the output unit projects at least one of the full-scale planned surgical line and the full-scale contour onto the body surface of the object with reference to a position of an ultrasonic probe used when the volume data is acquired.

10. The apparatus according to claim 1, wherein the output unit outputs at least one of the full-scale planned surgical line and the full-scale contour onto the body surface of the object by using a laser which does not damage a living body.

11. The apparatus according to claim 1, further comprising a control unit configured to control the data acquisition unit to make an ultrasonic beam irradiated from an ultrasonic probe used to acquire the volume data draw at least one of the full-scale planned surgical line and the full-scale contour onto one of a heat-sensitive sheet and a sound-sensitive sheet placed between the object and the ultrasonic probe.

12. A medical image diagnostic apparatus comprising:
a data acquisition unit configured to acquire volume data associated with the three-dimensional area including a predetermined region of an object;
a calculation unit configured to cut the volume data at at least one plane and calculate a full-scale contour of a slice of the predetermined region and a full-scale planned surgical line used for surgical operation of the predetermined region; and
an output unit configured to output at least one of the full-scale contour of the slice of the predetermined region and the full-scale planned surgical line.

13. The apparatus according to claim 12, wherein the calculation unit extracts a contour of the predetermined region included in the at least one plane, and calculates the full-scale contour of the predetermined region slice and the full-scale planned surgical line by using the extracted contour of the predetermined region.

14. The apparatus according to claim 12, wherein the calculation unit sets the plurality of planes, extracts the contour of the predetermined region included in each of the planes, and calculates the full-scale contour of the predetermined region slice by using a largest plane of the contours of the predetermined region extracted on the respective planes.

15. The apparatus according to claim 14, wherein the calculation unit sets the plurality of planes to be substantially parallel to each other.

16. The apparatus according to claim 12, wherein the calculation unit extracts the contour of the predetermined region in the volume data, and calculates the full-scale contour of the predetermined region slice and the full-scale planned surgical line by using the extracted contour at which the at least one plane is cut.

17. The apparatus according to claim 12, wherein the calculation unit calculates the full-scale planned surgical line by using the full-scale contour and a margin amount of a predetermined width.

18. The apparatus according to claim 12, wherein the output unit prints at least one of the full-scale planned surgical line and the full-scale contour onto a sheet configured to be pasted on the body surface of the object.

19. The apparatus according to claim 12, wherein the output unit projects at least one of the full-scale planned surgical line and the full-scale contour onto the body surface of the object.

20. The apparatus according to claim 19, wherein the output unit projects at least one of the full-scale planned surgical line and the full-scale contour onto the body surface of the object with reference to a position of an ultrasonic probe used when the volume data is acquired.

21. The apparatus according to claim 12, wherein the output unit outputs at least one of the full-scale planned surgical line and the full-scale contour onto the body surface of the object by using a laser which does not damage a living body.

22. An ultrasonic image processing apparatus comprising:
a calculation unit configured to cut volume data associated with a three-dimensional area including a predetermined region of an object which is obtained by ultrasonically scanning the three-dimensional area and calculate a full-scale contour of a slice of the predetermined region and a full-scale planned surgical line used for surgical operation of the predetermined region; and
an output unit configured to output at least one of the full-scale contour of the slice of the predetermined region and the full-scale planned surgical line.

23. A medical image processing apparatus comprising:
a calculation unit configured to cut volume data associated with a three-dimensional area including a predetermined region of an object at at least one plane and calculate a full-scale contour of a slice of the predetermined region and a full-scale planned surgical line used for surgical operation of the predetermined region; and
an output unit configured to output at least one of the full-scale contour of the slice of the predetermined region and the full-scale planned surgical line.

24. An ultrasonic diagnostic system comprising an ultrasonic diagnostic apparatus and an ultrasonic image processing apparatus,
the ultrasonic diagnostic apparatus comprising
a data acquisition unit configured to ultrasonically scan a three-dimensional area including a predetermined region of an object and acquire volume data associated with the three-dimensional area, and
the ultrasonic image processing apparatus comprising
a calculation unit configured to cut the volume data at at least one plane and calculate a full-scale contour of a slice of the predetermined region and a full-scale planned surgical line used for surgical operation of the predetermined region, and
an output unit configured to output at least one of the full-scale contour of the slice of the predetermined region and the full-scale planned surgical line.

25. A medical diagnostic system comprising a medical image diagnostic apparatus and a medical image processing apparatus,
the medical image diagnostic apparatus comprising
a data acquisition unit configured to acquire volume data associated with a three-dimensional area including a predetermined region of an object, and
the medical image processing apparatus comprising
a calculation unit configured to cut the volume data at at least one plane and calculate a full-scale contour of a slice of the predetermined region and a full-scale planned surgical line used for surgical operation of the predetermined region, and
an output unit configured to output at least one of the full-scale contour of the slice of the predetermined region and the full-scale planned surgical line.
